(19) United States
(12) Patent Application Publication
(10) Pub. No.: US 2007/0275432 A1
(43) Pub. Date: Nov. 29, 2007

(54) Means for a Quantitative Detection of Cytochrome C

(75) Inventors: Ludwig Baux, Paris (FR); Pierre Rustin, Paris (FR)

Correspondence Address: BUCHANAN, INGERSOLL & ROONEY PC, POST OFFICE BOX 1404, ALEXANDRIA, VA, 22313-1404, US

(73) Assignee: THERAPTOSIS S.A., Paris (FR)

(21) Appl. No.: 10/590,651

(22) PCT Filed: Feb. 24, 2005
(86) PCT No.: PCT/EP05/02577

§ 371 Date: May 31, 2007

Related U.S. Application Data
(60) Provisional application No. 60/547,564, filed on Feb. 26, 2004.

(51) Int. Cl. C12Q 1/26

(52) U.S. Cl. 435/25

(57) ABSTRACT

The invention relate to a method for detecting cytochrome c in a given biological sample, comprising: adding to said sample an efficient amount of two redox couples allowing for a cycling oxido-reduction of cytochrome c, said couples comprising an oxidizing agent consisting of cytochrome c oxidase enzyme and a reducing agent specific for cytochrome c with a reduced co-factor; measuring, by a biophysical system depending on the co-factor and allowing to distinguish the co-factor oxidized form from the reduced form, the oxidation of the co-factor which is oxidized during said cycling redox reaction; the amount of the co-factor oxidized form being correlated to the concentration of cytochrome c in the sample.

The invention relates to means for detecting cytochrome c release from mitochondria in a biological sample to be tested.

Mitochondria play a central role in the regulation of cellular apoptosis through release of proteins into cytosol.

Since the anti-apoptotic protein Bcl-2 was found to reside in mitochondria, scientists started to consider this organelle as important player in apoptosis. Two main findings reinforced the connection between Bcl-2, mitochondria and apoptosis: Bcl-2 was found to prevent the drop in mitochondrial membrane potential (Δψm) observed during TNF-induced apoptosis in many cell types. In the meantime, Newmeyer et al. discovered that a mitochondrial factor was required for the activation of caspases. This factor, released from mitochondria during apoptosis, was later identified as cytochrome c.

Two main apoptotic pathways have been described over the past few years: the death-receptor pathway and the mitochondrial pathway. The first is engaged by death receptors which, upon binding to their appropriate ligands, form a death-inducing signaling complex, resulting in pro-caspase-8 activation. In type I cells (T-Lymphocytes), caspase-8 cleaves downstream caspases that execute the cell. In type II (Hepatocytes), caspase-8 cleaves Bid, a BH3-only protein whose C-terminal fragment translocates to mitochondria to engage the mitochondrial pathway. Independently of death-receptor activation, the mitochondrial pathway can also be activated in response to a large number of death stimuli including DNA damage, topoisomerase inhibition or trophic-factor depletion. This process culminates in the release of mitochondrial proteins from the intermembrane space into the cytosol.

Wang's group showed that induction of caspase activity was dependent on the presence of cytochrome c released during the preparation of the cytosolic extract. Upon cytochrome c binding, Apaf-1 (apoptotic protease-activating factor 1) undergoes conformational changes and activates pro-caspase-9, leading to cell death.

Many laboratories over the world, interested in the mechanisms of apoptosis, are studying cytochrome c release in biological preparations (mitochondrial supernatants, cytosol extracts, etc) this release being a hallmark for apoptosis execution. Therefore, cytochrome c detection has a great interest for discovering pro- or anti-apoptotic drugs, integrated in screening strategies, alone or combined with other measurements (Δψm, mitochondrial swelling, etc).

This type of investigations is commonly conducted using immunodetection (for instance Western blotting or ELISA) as method of detection. However, Western blotting is a time consuming procedure (approximately 2 days) and a semi-quantitative method with poor accuracy. As a result, this method cannot be used for detailed analysis of the amount and kinetics of cytochrome c release under differing conditions, and cannot be used for drug screening. On the other hand, ELISA quantification requires plate coating, use of pre-set formats and is not really user-friendly because of the multiple washing stages.

HPLC has also been used for the quantitation of cytochrome c. Compared to Western blotting, the HPLC method is able to provide quantitative data. However samples have to be quantified sequentially and each quantification requires 20 minutes at least.

The inventors have found that by using cytochrome c-specific agents it was possible to determine cytochrome c concentration in different sub-cellular fractions by an enzymatic method.

The aim of the invention is first to provide a tool to accurately quantify cytochrome c concentration in biological samples. It is another object of the invention to provide a ready-made kit, for performing the described assay method. According to the invention, the method requires:
adding to the studied sample an efficient amount of two redox couples for a cycling oxydo-reduction of cytochrome c, said couples consisting in an oxidizing agent, cytochrome c oxidase enzyme (COX), and a reducing agent specific for cytochrome c with a co-factor,
measuring the oxidation of the co-factor which is oxidized during the redox cycle, the amount of the oxidative form thereof being correlated to the concentration of cytochrome c in the sample.

The cycling redox system which is generated enables, a highly specific and sensitive detection of cytochrome c. The reducing agent specifically reduces cytochrome c and this reduction is detected by monitoring the concomitant oxidation of said co-factor. To amplify the signal due to the co-factor oxidation, the oxidizing agent, cytochrome c oxidase, is added to the reaction medium to re-oxidize cytochrome c, allowing this cytochrome c to be used again by the reducing agent. The cycling redox system which is generated is strictly dependent on the presence of cytochrome c. Accordingly the amplification of the co-factor oxidation indicates that cytochrome c is present in the tested sample.

The oxidation of the co-factor is handily measured by a biophysical system depending on the co-factor and allowing to distinguish the oxidized form from the reduced form (for example but not limited to, absorbance measurement by molecular absorption spectrophotometry at 340 nm for NADH or NADPH detection).

In a preferred embodiment of the invention, the reducing agent is NADH-cytochrome c reductase or NADPH-cytochrome c reductase and the co-factor is NADH or NADPH respectively.

Advantageously, said measurement is compared to measurements of known concentrations of standard cytochrome c.

Said oxidizing and reducing agents and co-factors are, for example but not limited to, under liquid, dried or lyophilised form and obtained by purification of recombinant or natural compounds-or by chemical synthesis.

The detection may be performed on any biological sample suspected to contain cytochrome c, such as cellular extracts or organelles purified from primary cells, cell lines, tissues, blood, organs or tumors that may or may not have been submitted to stress, particularly apoptosis-inducing stress.

The detection is advantageously performed in supernatants obtained upon sedimenting mitochondria following incubation under various experimental conditions; or in cellular extracts obtained upon cytosol purification after cells incubation under various experimental conditions.

The invention also relates to a kit for detecting cytochrome c in sample to be tested. Such a kit, comprises:
two redox couples for a cycling oxido-reduction of cytochrome c, said couples consisting in an oxidizing agent, i.e. cytochrome c oxidase enzyme, and a reducing agent, specific for cytochrome c, using a reduced co-factor.

The reducing agent is advantageously a NADH-cytochrome c reductase or NADPH-cytochrome c reductase and the co-factor is NADH or NADPH respectively.

In the said kit, said agents are, for example but not limited to, under liquid, dried or lyophilised form and obtained by purification of recombinant or natural compounds or by chemical synthetis.

Optionally, said kit further comprises a buffer.

Said kit also comprises standard cytochrome c as a reference.

Said means are advantageous substitutes to immuno-assay, HPLC detection or Western blotting in order to detect cytochrome c in biological samples.

The present invention is further illustrated by the following examples and figures, which respectively represent:

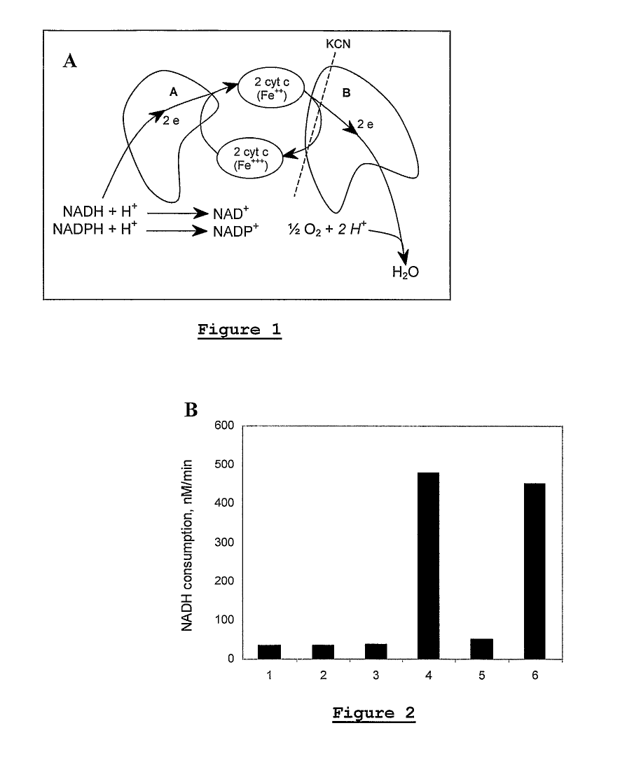

FIG. 1: Cycling enzyme assay for the detection of cytochrome c;

FIG. 2: NAD(P)H oxidation is dependent on the simultaneous presence of the various components of the enzymatic cycle and is fully blocked upon cytochrome c oxidase inhibition;

FIG. 3: At constant cytochrome c concentration, the rate of the cycling reaction is dependent on the amount of added enzymes;

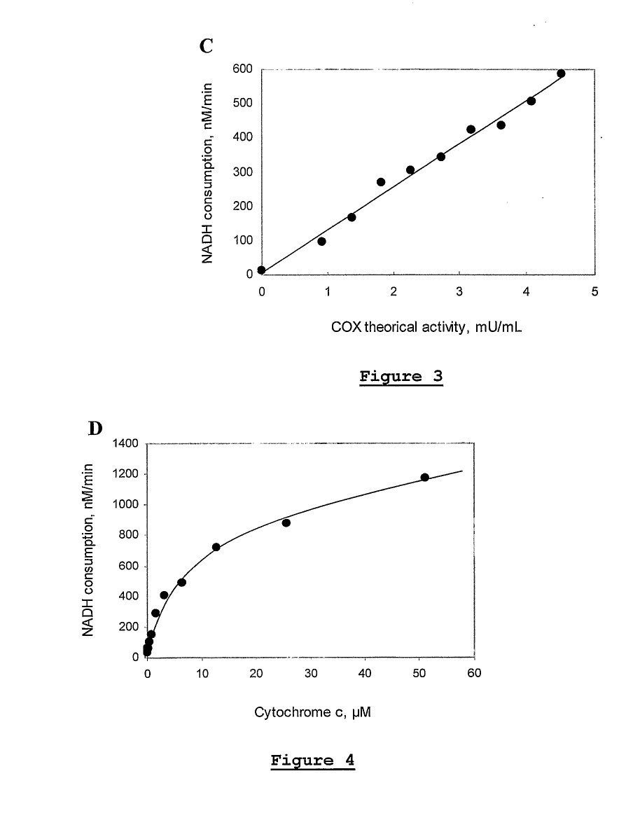

FIG. 4: At saturating enzyme concentrations, the rate of the cycling assay only depends on cytochrome c concentration;

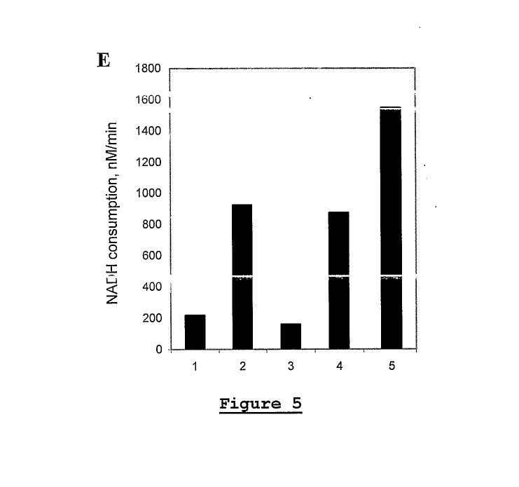

FIG. 5: Saturating enzyme concentration permits to detect low concentration of cytochrome c in mitochondrial supernatants.

In the current embodiment of the invention, measurements are performed in transparent flat-bottom 96-well microplates in a final volume of 220 microliter. Enzymes, prepared enzyme buffer (10 mM Tris-HCl, pH 7.0, 250 mM saccharose), were added (20 microliter per well) to 180 microliter assay buffer (10 mM Tris-HCl, pH 7.0, 120 mM KCl, 300 μM NADH or NADPH). The reaction is started by adding 20 microliter of either purified cytochrome c or desired sample to obtain a final volume of 220 microliter.

NADH or NADPH oxidation is spectrophotometrically measured at 340 nm, as both NADH and NADPH absorb at this wavelength while NAD⁺ and NADP⁺ do not. For the determination of the rate of co-factor oxidation, absorbance is monitored in kinetic mode for 30 minutes at room temperature.

$$Rate of NA DH consumption ( M / min ) = \frac{( {DO}_{t = 180 s} - {DO}_{t = 1800 s} ) * 60}{( 1800 - 180 ) * 6230}$$

Stock solution of cytochrome c and NADH or NADPH are prepared in distilled water.

Cycling enzyme assay for the detection of cytochrome c (FIG. 1).

Enzyme A is a cytochrome c reductase that catalyses cytochrome c reduction and concomitant co-factor (NADH or NADPH) oxidation. Oxidized cytochrome c is thus reduced by the reductase. Enzyme B is the cytochrome c oxidase: it oxidizes reduced cytochrome c and transfers electron to molecular oxygen. The presence of both enzymes allows for a redox cycle using cytochrome c. The rate of cycling becomes limited by cytochrome c in the presence of an excess of both enzymes. The rate of NAD(P)H oxidation is then directly proportional to the amount of available cytochrome c. Cyanide (KCN) is a cytochrome c oxidase inhibitor.

NAD(P)H oxidation is dependent on the simultaneous presence of the various components of the enzymatic cycle and is fully blocked upon cytochrome c oxidase inhibition. Measurements are performed by absorption spectrophotometry at 340 nm (FIG. 2).

Complete medium: 300 μM NADH, 300 μU NADH-cytochrome c reductase, 300 μU cytochrome c oxidase, 2 μM cytochrome c; 1. NADH only; 2. Complete medium without cytochrome c oxidase; 3. Complete medium without cytochrome c; 4. Complete medium without NADH-cytochrome c reductase; 5. Complete medium added with 500 μM KCN as to inhibits cytochrome c oxidase; 6. Complete medium; noticeably, the absence of any of the components hampers cycle operation, except for the absence of added NADH-cytochrome c reductase, due to the contamination of cytochrome c oxidase preparation by NADH-cytochrome c reductase.

At constant cytochrome c concentration, the rate of the cycling reaction is dependent on the amount of added enzymes. Measurements are performed by absorption spectrophotometry at 340 nm (FIG. 3).

In the low range of enzyme concentrations, the oxidation of NADH (300 μM) correlates with enzyme concentration. In the selected example, NADH-cytochrome c reductase is in excess compared to cytochrome c oxidase, and the rate of the reaction is dependent on this latter enzyme. A large excess of both enzymes is therefore required to avoid any interference that may result from enzymes potentially added with studied samples.

At saturating enzyme concentrations, the rate of the cycling assay only depends on cytochrome c concentration. Measurements are performed by absorption spectrophotometry at 340 nm (FIG. 4).

At high enzyme concentrations (240 μU NADH-cytochrome c reductase, 240 μU cytochrome c oxidase/well), NADH (300 μM) is oxidized proportionally to the cytochrome c concentration. For low cytochrome c concentrations, a linear relationship exists between the rate of NADH consumption and the cytochrome c concentration. It makes this reaction a simple and convenient method to quantify cytochrome c.

Saturating enzyme concentration permits to detect low concentration of cytochrome c in mitochondrial supernatants. Measurements are performed by absorption spectrophotometry at 340 nm (FIG. 5).

Preparation and incubation of mitochondria: Mouse liver mitochondria were prepared by a standard procedure with slight modifications. Briefly, the minced liver was homogenized in medium A (0.3 M saccharose, 0.2 mM EGTA and 5 mM TES, pH 7.2). Homogenates were centrifuged at low speed (760 g, 10 min, at 4° C.), collected supernatants diluted in medium A and further centrifuged (8740 g, 10 min at 4° C.). Washed mitochondria were layered on top of two successive Percoll gradients, consisting of three layers of 18%, 30% and 60% (w/v) Percoll in medium B (0.3 M saccharose, 0.2 mM EGTA, 10 mM TE; pH 6.9). After centrifugation (8740 g, 10 min), the fraction containing intact mitochondria was collected from the 30%/60% interface, washed with medium A (8740 g, 10 min) and the pellet resuspended in 500 μL medium A. Protein concentration was determined by BCA assay.

Purified mitochondria (4 mg protein/mL) were then incubated in medium C (0.2 M saccharose, 5 mM succinate, 10 μM EGTA, 1 mM H₃PO₄, 2 μM Rotenone and 10 mM Tris-MOPS; pH 7.4) for 30 min at room temperature with 500 μM calcium chloride or 5 μg/mL alamethicin as inducers of cytochrome c release.

Preparation of samples: Treated mitochondria were centrifuged (6800 g, 10 min at 4° C.) and supernatants were about 20 fold-concentrated on 10,000 Da concentrator microtubes for 15 min at 12,000 g, room temperature. Each sample (20 microliter) was added to 200 microliter reaction solution (300 μM NADH in 180 μL assay buffer, 1 mU NADH-cytochrome c reductase in 10 μL enzyme buffer, 1 mU cytochrome c oxidase in 10 μL enzyme buffer) ; 1. Distilled water; 2. Purified cytochrome c 100 nM; 3. Supernatant of intact purified mitochondria; 4. Supernatant from calcium-treated mitochondria; 5. Supernatant from alamethicin-treated mitochondria. This set of experiments establishes that the proposed cycling enzyme assay is sensitive enough as to quantify cytochrome c released from mitochondrial preparations.

1. A method for detecting cytochrome c in a given biological sample, comprising: adding to said sample an efficient amount of two redox couples allowing for a cycling oxido-reduction of cytochrome c, said couples comprising an oxidizing agent consisting of cytochrome c oxidase enzyme and a reducing agent specific for cytochrome c with a reduced co-factor; measuring, by a biophysical system depending on the co-factor and allowing to distinguish the co-factor oxidized form from the reduced form, the oxidation of the co-factor which is oxidized during said cycling redox reaction; the amount of the co-factor oxidized form being correlated to the concentration of cytochrome c in the sample.

2. The method of claim 1, wherein said measurement is compared to measurements performed with standard cytochrome c.

3. The method of claim 1, wherein the reducing agent is NADH-cytochrome c reductase or NADPH-cytochrome c reductase and the reduced co-factor is NADH or NADPH respectively.

4. The method of claim 1, wherein the co-factor is detected by absorption spectrophotometry at 340 nm.

5. The method of claim 1, wherein said agents are under liquid, dried or lyophilised form and obtained by purification of recombinant or natural compounds or by chemical synthesis.

6. The method of claim 1, optimized for any new screening protocol or adaptaded to any existing screening procedure.

7. A kit for detecting cytochrome c in sample to be tested, comprising two redox couples for a cycling oxido-reduction of cytochrome c, said couples comprising an oxidizing agent consisting of cytochrome c oxidase enzyme and a reducing agent specific for cytochrome c with a reduced co-factor.

8. The kit of claim 7, wherein the reducing agent is NADH-cytochrome c reductase and the co-factor is NADH.

9. The kit of claim 7, wherein the reducing agent is NADPH-cytochrome c reductase and the co-factor is NADPH.

10. The kit of claim 7, further comprising cytochrome c as a reference standard.

11. The kit of claim 7, further comprising a buffer.

12. The kit of claim 7, wherein said agents are under liquid, dried or lyophilised. form, and obtained by purification of recombinant' or natural compounds or by chemical synthesis.

13. The kit of claim 7, defined for laboratory research only.

14. The kit of claim 7, defined for diagnostic use.

15. The kit of claim 7, optimized 96-well microplates, 384-well microplates, 1 mL cuvettes.

16. The kit of claim 7, optimized for detecting cytochrome c in mitochondrial supernatants.

17. The kit of claim 7, optimized for detecting cytochrome c in cytosol extracts.

18. The kit of claim 7, optimized for detecting cytochrome c in any other biological sample expected to contain cytochrome c.

19. The kit of claim 18, with reagents supplied for the preparation of mitochondrial and/or cytosolic fractions.

20. The kit of claim 19, with methodology for the preparation of mitochondrial and/or cytosolic fractions.
